(19) United States
(12) Patent Application Publication
(10) Pub. No.: US 2011/0034384 A1
(43) Pub. Date: Feb. 10, 2011

(54) COMBINATION VEGFR2 THERAPY WITH mTOR INHIBITORS

(75) Inventor: Irvith M. Carvajal, Brighton, MA (US)

Correspondence Address: ROPES & GRAY LLP;IPRM - Floor 43, Prudential Tower, 800 Boylston Street, Boston, MA, 02199-3600, US

(73) Assignee: Bristol-Myers Squibb Company, Princeton, NJ (US)

(21) Appl. No.: 12/745,045

(22) PCT Filed: Nov. 24, 2008
(86) PCT No.: PCT/US08/13091

§ 371 Date: Oct. 21, 2010

Related U.S. Application Data
(60) Provisional application No. 61/004,666, filed on Nov. 28, 2007.

(51) Int. Cl. A61K 38/39; A61P 35/00

(52) U.S. Cl. 514/9.3

(57) ABSTRACT

The present disclosure relates to improved methods of treating neoplastic disorders by combining VEGFR2 specific inhibitor treatment with mTOR inhibitors. The present disclosure also relates to methods of preventing the development of VEGFR2 resistance.

### CROSS REFERENCE TO RELATED APPLICATION

This application claims the benefit of U.S. Provisional Application Ser. No. 61/004,666 filed Nov. 28, 2007, the entire contents of which are incorporated herein by reference.

### BACKGROUND OF THE INVENTION

Angiogenesis is the process by which new blood vessels are formed from pre-existing capillaries or post capillary venules; it is an important component of many physiological processes. In cancer, tumor released cytokines or angiogenic factors stimulate vascular endothelial cells by interacting with specific cell surface receptors. The activated endothelial cells secrete enzymes that degrade the basement membrane of the vessels, allowing invasion of the endothelial cells into the tumor tissue. Once situated, the endothelial cells differentiate to form new vessel offshoots of pre-existing vessels. The new blood vessels provide nutrients to the tumor, facilitating further growth, and also provide a route for metastasis.

Additional therapies are needed to treat cancer, in particular to inhibit angiogenesis associated with cancer.

### SUMMARY OF THE INVENTION

One aspect of the application provides for a method of treating a subject afflicted with a neoplasm by administering to the subject at least one VEGFR2 specific inhibitor together or in parallel with at least one mTOR inhibitor in amounts that together are effective to treat the neoplasm. In some embodiments, the mTOR inhibitor is temsilolimus.

Another aspect of the application provides for a method of reducing the severity, delaying the onset, or preventing the development of VEGFR2 resistance in a subject afflicted with a neoplasm by administering at least one VEGFR2 specific inhibitor together or in parallel with at least one mTOR inhibitor. In some embodiments, the development of VEGFR2 resistance is delayed by at least one week.

The neoplasm may be any abnormal proliferation of cells benign or malignant. In some embodiments, the neoplasm is a solid tumor. In some embodiments, the neoplasm is a cancer. In some embodiments, the neoplasm is dependent on angiogenesis for growth or survival.

In some embodiments, the VEGFR2 specific inhibitor and the mTOR inhibitor are administered sequentially. In some embodiments, the inhibitors are administered together.

In some embodiments, the VEGFR2 specific inhibitor is selected from an antibody or a fibronectin based scaffold protein.

In some embodiments, the VEGFR2 specific inhibitor is an antibody-like protein comprising a tenth fibronectin type III domain (¹⁰Fn3), wherein the amino acid sequence of the ¹⁰Fn3 is altered in one or more of the BC, DE, or FG loops, relative to the naturally occurring human ¹⁰Fn3 as depicted in SEQ ID NO: 1. In some embodiments, the VEGFR2 binding ¹⁰Fn3 comprises a BC loop having the amino acid sequence set for in residues 16-23 SEQ ID NO: 4, a DE loop having the amino acid sequence set for in residues 45-49 of SEQ ID NO: 4, and an FG loop having the amino acid sequence set for in residues 70-80 of SEQ ID NO: 4. In some embodiments, the VEGFR2 binding ¹⁰Fn3 has an amino acid sequence at least 70, 80, 90, 95, 98, 99, or 100% identical to SEQ ID NO: 4.

In some embodiments, the VEGFR2 specific inhibitor is a polypeptide comprising an amino acid sequence at least 70, 80, 90, 95, 98, 99, or 100% identical to any one of SEQ ID NOS: 2-59.

In some embodiments, methods are provided comprising conjointly administering to a patient in need thereof, temsilolimus and a ¹⁰Fn3 polypeptide comprising a BC loop having the amino acid sequence set for in residues 16-23 SEQ ID NO: 4, a DE loop having the amino acid sequence set for in residues 45-49 of SEQ ID NO: 4, and an FG loop having the amino acid sequence set for in residues 70-80 of SEQ ID NO: 4 and having an amino acid sequence at least 70, 80, 90, 95, 98, 99, or 100% identical to SEQ ID NOS: 4.

### BRIEF DESCRIPTION OF THE DRAWINGS

FIG. 1 Colo205 tumor growth is equally inhibited by Comp-I and Temsirolimus. Their combination significantly enhances tumor growth inhibition.

FIG. 2. Sustained tumor growth inhibition by the combination of Comp-I and Temsirolimus is observed after suspension of treatment compared to either monotherapy.

FIG. 3. The combination of Comp-I and Temsirolimus synergistically enhances the antitumor activities of either agent by significantly inhibiting tumor doubling time.

FIG. 4. Temsirolimus added to Comp-I or Bevacizumab treatment at Day 17.

FIG. 5. Temsirolimus added to Comp-I or Bevacizumab treatment at Day 9.

FIG. 6. Temsirolimus added to Comp-I or Bevacizumab treatment at Day 23.

### DETAILED DESCRIPTION OF THE INVENTION

### Definitions

By a “polypeptide” is meant any sequence of two or more amino acids, regardless of length, post-translation modification, or function. “Polypeptide,” “peptide,” and “protein” are used interchangeably herein. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D).

When used herein, “fibronectin-based scaffold protein” refers to polypeptides based on a fibronectin type III domain (Fn3). An example of fibronectin-based scaffold proteins are Adnectins™ (Adnexus Therapeutics, Inc.). Fibronectin is a large protein which plays essential roles in the formation of extracellular matrix and cell-cell interactions; it consists of many repeats of three types (types I, II, and III) of small domains (Baron et al., 1991). Fn3 itself is the paradigm of a large subfamily which includes portions of cell adhesion molecules, cell surface hormone and cytokine receptors, chaperoning, and carbohydrate-binding domains. For reviews see Bork & Doolittle, Proc Natl Acad Sci USA. 1992 Oct. 1; 89(19):8990-4; Bork et al., J Mol. Biol. 1994 Sep. 30; 242(4):309-20; Campbell & Spitzfaden, Structure. 1994 May 15; 2(5):333-7; Harpez & Chothia, J Mol. Biol. 1994 May 13; 238(4):528-39).

Preferably, the fibronectin-based scaffold protein is a “¹⁰Fn3” scaffold, by which is meant a polypeptide variant based on the tenth module of the human fibronectin type III protein in which one or more of the solvent accessible loops has been randomized or mutated, particularly one or more of the three loops identified as the BC loop (amino acids 23-30), DE loop (amino acids 52-56) and FG loop (amino acids 77-87) (the numbering scheme is based on the sequence on the tenth Type III domain of human fibronectin, with the amino acids Val-Ser-Asp representing amino acids numbers 1-3). The amino acid sequence of the wild-type tenth module of the human fibronectin type III domain is:

(SEQ ID NO: 1)
VSDVPRDLEVVAATPTSLLISWDAPAVTVRYYRITYGETGGNSPVQEFTV
PGSKST ATISGLKPGVDYTITVYAVTGRGDSPASSKPISINYRT.

Preferably, fibronectin-based scaffold proteins are based on SEQ ID NO:1.

A variety of mutant 10Fn3 scaffolds have been reported. In one aspect, one or more of Asp 7, Glu 9, and Asp 23 is replaced by another amino acid, such as, for example, a non-negatively charged amino acid residue (e.g., Asn, Lys, etc.). These mutations have been reported to have the effect of promoting greater stability of the mutant 10Fn3 at neutral pH as compared to the wild-type form (See, PCT Publication No. WO02/04523). A variety of additional alterations in the 10Fn3 scaffold that are either beneficial or neutral have been disclosed. See, for example, Batori et al., Protein Eng. 2002 Dec.; 15(12):1015-20; Koide et al., Biochemistry 2001 Aug. 28; 40(34):10326-33.

Both the variant and wild-type ¹⁰Fn3 proteins are characterized by the same structure, namely seven beta-strand domain sequences (designated A through G and six loop regions (AB loop, BC loop, CD loop, DE loop, EF loop, and FG loop) which connect the seven beta-strand domain sequences. The beta strands positioned closest to the N- and C-termini may adopt a beta-like conformation in solution. In SEQ ID NO:1, the AB loop corresponds to residues 15-16, the BC loop corresponds to residues 22-30, the CD loop corresponds to residues 39-45, the DE loop corresponds to residues 51-55, the EF loop corresponds to residues 60-66, and the FG loop corresponds to residues 76-87. As shown in Figures, the BC loop, DE loop, and FG loop are all located at the same end of the polypeptide. Similarly, immunoglobulin scaffolds tend to have at least seven beta or beta-like strands, and often nine beta or beta-like strands. Fibronectin-based scaffold proteins can include other Fn3 type fibronectin domains as long as they exhibit useful activities and properties of ¹⁰Fn3 type domains.

“VEGFR-2 binding protein” refers to polypeptides that bind specifically to VEGFR-2 relative to other related proteins from the same species. By “specifically binds” is meant a polypeptide that recognizes and interacts with a target protein (e.g., VEGFR-2) but that does not substantially recognize and interact with other molecules in a sample, for example, a biological sample. In preferred embodiments a polypeptide of the invention will specifically bind a VEGFR-2 with a K_{D} at least as tight as 500 nM. Preferably, the polypeptide will specifically bind a VEGFR-2 with a K_{D} of 1 pM to 500 nM, more preferably 1 pM to 100 nM, more preferably 1 pM to 10 nM, and most preferably 1 pM to 1 nM or lower.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the time to disease progression (TTP) and/or determining the response rates (RR).

As used herein, the term “synergistic” refers to a combination of two monotherapies, which is more effective than the additive effects of the therapies. A synergistic effect of a combination of monotherapies permits the use of lower dosages of one or more of the therapies and/or less frequent administration of said therapies to a subject with a neoplastic disorder. The ability to utilize lower dosages of a therapy and/or to administer said therapy less frequently reduces the toxicity associated with the administration of said therapy to a subject without reducing the efficacy of said therapy in the prevention, management or treatment of a neoplastic disorder. In addition, a synergistic effect can result in improved efficacy of agents in the prevention, management or treatment of a neoplastic disorder. Finally, a synergistic effect of a combination of two monotherapies may avoid or reduce adverse or unwanted side effects associated with the use of either therapy alone, such as, for example resistance.

As used herein, “resistance” or “resistant” refers to a neoplasm having cells that express a resistant mutant form of VEGF receptor or cells that overexpress a VEGF receptor. Resistance includes other known mechanisms of resistance (e.g., efflux pump in resistant cells, upregulation of ligand). The net effect of the resistance is that the use of the VEGFR2 inhibitor as a monotherapy for the treatment of the resistant cells is less effective than when used to treat a non-resistant cells.

By “treating” is meant to slow the spreading of the cancer, to slow the cancer's growth, to kill or arrest cancer cells that may have spread to other parts of the body from the original tumor, to relieve symptoms caused by the cancer, or to prevent cancer. The symptoms to be relieved using the combination therapies described herein include pain, and other types of discomfort.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

### Overview

The application relates, in part, on the surprising discovery that the combination of a VEGFR2 specific inhibitor and an mTOR inhibitor results in a synergistic therapeutic effect in an in vivo tumor model. The application provides novel methods of treatment and combination therapies to treat neoplasms, in particular angiogenesis dependent neoplasms. The novel treatment regimes comprise the administration of at least one VEGFR2 specific inhibitor and at least one mTOR inhibitor.

VEGF, acting through its cognate receptors, can function as an endothelial specific mitogen during angiogenesis. In addition, there is substantial evidence that VEGF and VEGFRs are up-regulated in conditions characterized by inappropriate angiogenesis, such as cancer.

The macrolide fungicide rapamycin, a natural product with anti-tumor properties, is also capable of inhibiting signal transduction pathways that are necessary for the proliferation of cells. Rapamycin binds intracellularly to the immunophilin FK506 binding protein 12 (FKBP12), and the resultant complex inhibits the serine protein kinase activity of mammalian target of rapamycin (mTOR). The inhibition of mTOR, in turn, blocks signals to at least two separate downstream pathways which control the translation of specific mRNAs required for cell proliferation.

The combination of an mTOR inhibitor and a VEGFR2 specific inhibitor provides an improved method of treatment for neoplasms.

### VEGFR2 Specific Inhibitors

VEGFR2 specific inhibitors useful in the present invention may be any protein or small molecule that binds VEGFR2 and inhibits or reduces one or more VEGFR2 biological functions.

Examples of VEGFR2 specific inhibitors include antibodies, such as heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine.

According to one aspect of the invention, a single domain antibodies as used herein is a naturally occurring single domain antibody known as heavy chain antibody devoid of light chains. Such single domain antibodies are disclosed in WO 94/04678 for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, dromedary, llama, vicuna, alpaca and guanaco. Other species besides Camelidae may produce heavy chain antibodies naturally devoid of light chain; such VHHs are within the scope of the invention.

VHHs, according to the present invention, and as known to the skilled in the art are heavy chain variable domains derived from immunoglobulins naturally devoid of light chains such as those derived from Camelidae as described in WO 94/04678 (and referred to hereinafter as VHH domains or nanobodies). VHH molecules are about 10 times smaller than IgG molecules. They are single polypeptides and very stable, resisting extreme pH and temperature conditions. Moreover, they are resistant to the action of proteases which is not the case for conventional antibodies. Furthermore, in vitro expression of VHHs produces high yield, properly folded functional VHHs. In addition, antibodies generated in Camelids will recognize epitopes other than those recognized by antibodies generated in vitro through the use of antibody libraries or via immunization of mammals other than Camelids (WO9749805). As such, anti VEGFR2VHH's may interact more efficiently with VEGFR2 than conventional antibodies, thereby blocking its interaction with the VEGF ligand(s) more efficiently. Since VHH's are known to bind into ‘unusual’ epitopes such as cavities or grooves (WO97/49805), the affinity of such VHH's may be more suitable for therapeutic treatment.

Another example of a VEGFR2 specific inhibitor is anti-VEGFR-2 consisting of a sequence corresponding to that of a Camelidae VHH directed towards VEGFR-2 or a closely related family member. The invention also relates to a homologous sequence, a function portion or a functional portion of a homologous sequence of said polypeptide. The invention also relates to nucleic acids capable of encoding said polypeptides. A single domain antibody of the present invention may be directed against VEGFR-2 or a closely related family member.

The present invention further relates to single domain antibodies of VHH belonging to a class having human-like sequences. One such class is characterized in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 and a tryptophan at position 103, according to the Kabat numbering. As such, polypeptides belonging to this class show a high amino acid sequence homology to human VH framework regions and said polypeptides might be administered to a human directly without expectation of an unwanted immune response therefrom, and without the burden of further humanisation.

Another human-like class of Camelidae single domain antibodies has been described in PCT Publication No. WO03/035694 and contain the hydrophobic FR2 residues typically found in conventional antibodies of human origin or from other species, but compensating this loss in hydrophilicity by the charged arginine residue on position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanization. The invention also relates to nucleic acids capable of encoding said polypeptides. Polypeptides may include the full length Camelidae antibodies, namely Fc and VHH domains.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)₂ fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

Various techniques have been developed for the production of antibody fragments that may be used to make antibody fragments used in the invention. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

In one aspect, the VEGFR2 antibody is CDP-791 (UCB). In another aspect, the VEGFR2 antibody is IMC-1121b (ImClone Systems). In yet another aspect, the VEGFR2 inhibitor is AVE-005 (VEGF trap, Regeneron Pharmaceuticals).

Other examples of VEGFR2 specific inhibitors include moieties such as affibodies, afflins, anticalins, avimers, DARPins, microbodies, trans-bodies; or inhibitors that are derived from lipocalins, ankyrins, tetranectins, C-type lectin, Protein A, gamma-crystalline, cysteine knots, and transferrin.

In some embodiments, the VEGFR2 specific inhibitors comprise an immunoglobulin-like domain. One, two, three or more loops of the immunoglobulin-like domain may participate in binding to VEGFR-2. A preferred immunoglobulin-like domain is a fibronectin type III (Fn3) domain. Such domain may comprise, in order from N-terminus to C-terminus, a beta or beta-like strand, A; a loop, AB; a beta strand, B; a loop, BC; a beta strand C; a loop CD; a beta strand D; a loop DE; a beta strand F; a loop FG; and a beta or beta-like strand G. Any or all of loops AB, BC, CD, DE, EF and FG may participate in VEGFR-2 binding, although preferred loops are BC, DE and FG.

A preferred Fn3 domain is an Fn3 domain derived from human fibronectin, particularly the 10^{th} Fn3 domain of fibronectin, referred to as ¹⁰Fn3. It should be noted that none of VEGFR-2 binding polypeptides disclosed herein have an amino acid sequence that is identical to native ¹⁰Fn3; the sequence has been modified to obtain VEGFR-2 specific inhibitors, but proteins having the basic structural features of ¹⁰Fn3, and particularly those retaining recognizable sequence homology to the native ¹⁰Fn3 are nonetheless referred to herein as “¹⁰Fn3 polypeptides”. This nomenclature is similar to that found in the antibody field where, for example, a recombinant antibody V_{L} domain generated against a particular target protein may not be identical to any naturally occurring V_{L} domain but nonetheless the protein is recognizably a V_{L} protein.

¹⁰Fn3 are structurally and functionally analogous to antibodies, specifically the variable region of an antibody. While ¹⁰Fn3 domains may be described as “antibody mimics” or “antibody-like proteins”, they do offer a number of advantages over conventional antibodies. In particular, they exhibit better folding and thermostability properties as compared to antibodies, and they lack disulphide bonds, which are known to impede or prevent proper folding under certain conditions.

A ¹⁰Fn3 polypeptide may be at least 60%, 65%, 70%, 75%, 80%, 85%, or 90% identical to the human ¹⁰Fn3 domain (SEQ ID NO: 1). Much of the variability will generally occur in one or more of the loops. Each of the beta or beta-like strands of a ¹⁰Fn3 polypeptide may consist essentially of an amino acid sequence that is at least 80%, 85%, 90%, 95% or 100% identical to the sequence of a corresponding beta or beta-like strand of SEQ ID NO: 1, provided that such variation does not disrupt the stability of the polypeptide in physiological conditions. A ¹⁰Fn3 polypeptide may have a sequence in each of the loops AB, CD, and EF that consists essentially of an amino acid sequence that is at least 80%, 85%, 90%, 95% or 100% identical to the sequence of a corresponding loop of SEQ ID NO:1. In many instances, any or all of loops BC, DE, and FG will be poorly conserved relative to SEQ ID NO:1. For example, all of loops BC, DE, and FG may be less than 20%, 10%, or 0% identical to their corresponding loops in SEQ ID NO:1.

Working examples of VEGFR-2 specific inhibitors were generated as described in PCT Publication No. WO 2005/056764, which is hereby incorporated by reference.

Sequences of preferred VEGFR-2 binding ¹⁰Fn3 polypeptides useful for the invention are as follows:

SEQ ID NO: 2
VSDVPRDLEVVAATPTSLLISWRHPHFPTRYYRITYGETGGNSPVQEFTV
PLQPPTATISGLKPGVDYTITVYAVTEGPNERSLFIPISINYRT
SEQ ID NO: 3
EVVAATPTSLLISWRHPHFPTRYYRITYGETGGNSPVQEFTVPLQPPTAT
ISGLKPGVDYTITVYAVTEGPNERSLFIPISINYRT
SEQ ID NO: 4
GEVVAATPTSLLISWRHPHFPTRYYRITYGETGGNSPVQEFTVPLQPPTA
TISGLKPGVDYTITVYAVTDGRNGRLLSIPISINYRTEIDKPCQ
SEQ ID NO: 5
EVVAATPTSLLISWRHPHFPTRYYRITYGETGGNSPVQEFTVPLQPPTAT
ISGLKPGVDYTITVYAVTDGRNGRLLSIPISINYRT
SEQ ID NO: 6
EVVAATPTSLLISWRHPHFPTRYYRITYGETGGNSPVQEFTVPLQPPTAT
ISGLKPGVDYTITGYAVTMGLYGHELLTPISINYRT
SEQ ID NO: 7
EVVAATPTSLLISWRHPHFPTRYYRITYGETGGNSPVQEFTVPLQPPTAT
ISGLKPGVDYTITGYAVTDGENGQFLLVPISINYRT
SEQ ID NO: 8
EVVAATPTSLLISWRHPHFPTRYYRITYGETGGNSPVQEFTVPLQPPTAT
ISGLKPGVDYTITGYAVTMGPNDNELLTPISINYRT
SEQ ID NO: 9
EVVAATPTSLLISWRHPHFPTRYYRITYGETGGNSPVQEFTVPLQPPTAT
ISGLKPGVDYTITGYAVTAGWDDHELFIPISINYRT
SEQ ID NO: 10
EVVAATPTSLLISWRHPHFPTRYYRITYGETGGNSPVQEFTVPLQPPTAT
ISGLKPGVDYTITGYAVTSGHNDHMLMIPISINYRT
SEQ ID NO: 11
EVVAATPTSLLISWRHPHFPTRYYRITYGETGGNSPVQEFTVPLQPPTAT
ISGLKPGVDYTITGYAVTAGYNDQILMTPISINYRT
SEQ ID NO: 12
EVVAATPTSLLISWRHPHFPTRYYRITYGETGGNSPVQEFTVPLQPPTAT
ISGLKPGVDYTITGYAVTFGLYGKELLIPISINYRT
SEQ ID NO: 13
EVVAATPTSLLISWRHPHFPTRYYRITYGETGGNSPVQEFTVPLQPPTAT
ISGLKPGVDYTITGYAVTTGPNDRLLFVPISINYRT
SEQ ID NO: 14
EVVAATPTSLLISWRHPHFPTRYYRITYGETGGNSPVQEFTVPLQPPTAT
ISGLKPGVDYTITGYAVTDVYNDHEIKTPISINYRT
SEQ ID NO: 15
EVVAATPTSLLISWRHPHFPTRYYRITYGETGGNSPVQEFTVPLQPPTAT
ISGLKPGVDYTITGYAVTDGKDGRVLLTPISINYRT
SEQ ID NO: 16
EVVAATPTSLLISWRHPHFPTRYYRITYGETGGNSPVQEFTVPLQPPTAT
ISGLKPGVDYTITGYAVTEVHHDREIKTPISINYRT
SEQ ID NO: 17
EVVAATPTSLLISWRHPHFPTRYYRITYGETGGNSPVQEFTVPLQPPTAT
ISGLKPGVDYTITGYAVTQAPNDRVLYTPISINYRT
SEQ ID NO: 18
EVVAATPTSLLISWRHPHFPTRYYRITYGETGGNSPVQEFTVPLQPPTAT
ISGLKPGVDYTITGYAVTREENDHELLIPISINYRT
SEQ ID NO: 19
EVVAATPTSLLISWRHPHFPTRYYRITYGETGGNSPVQEFTVPLQPPTAT
ISGLKPGVDYTITGYAVTVTHNGHPLMTPISINYRT
SEQ ID NO: 20
EVVAATPTSLLISWRHPHFPTRYYRITYGETGGNSPVQEFTVPLQPPTAT
ISGLKPGVDYTITGYAVTLALKGHELLTPISINYRT
SEQ ID NO: 21
VSDVPRDLEVVAATPTSLLISWRHPHFPTRYYRITYGETGGNSPVQEFTV
PLQPPTATISGLKPGVDYTITGYAVTVAQNDHELITPISINYRT
SEQ ID NO: 22
VSDVPRDL/QEVVAATPTSLLISWRHPHFPTRYYRITYGETGGNSPVQEF
TVPLQPPAATISGLKPGVDYTITGYAVTMAQSGHELFTPISINYRT
SEQ ID NO: 23
EVVAATPTSLLISWRHPHFPTRYYRITYGETGGNSPVQEFTVPLQPPTAT
ISGLKPGVDYTITGYAVTVERNGRVLMTPISINYRT
SEQ ID NO: 24
EVVAATPTSLLISWRHPHFPTRYYRITYGETGGNSPVQEFTVPLQPPTAT
ISGLKPGVDYTITGYAVTVERNGRHLMTPISINYRT
SEQ ID NO: 25
EVVAATPTSLLISWRHPHFPTRYYRITYGETGGNSPVQEFTVPLQPPTAT
ISGLKPGVDYTITGYAVTLERNGRELMTPISINYRT
SEQ ID NO: 26
EVVAATPTSLLISWRHPHFPTRYYRITYGETGGNSPVQEFTVPLQPPTAT
ISGLKPGVDYTITGYAVTEERNGRTLRTPISINYRT
SEQ ID NO: 27
EVVAATPTSLLISWRHPHFPTRYYRITYGETGGNSPVQEFTVPLQPPTAT
ISGLKPGVDYTITGYAVTVERNDRVLFTPISINYRT
SEQ ID NO: 28
EVVAATPTSLLISWRHPHFPTRYYRITYGETGGNSPVQEFTVPLQPPTAT
ISGLKPGVDYTITGYAVTVERNGRELMTPISINYRT
SEQ ID NO: 29
EVVAATPTSLLISWRHPHFPTRYYRITYGETGGNSPVQEFTVPLQPPTAT
ISGLKPGVDYTITGYAVTLERNGRELMVPISINYRT
SEQ ID NO: 30
EVVAATPTSLLISWRHPHFPTRYYRITYGETGGNSPVQEFTVPLQPPTAT
ISGLKPGVDYTITGYAVTDGRNDRKLMVPISINYRT
SEQ ID NO: 31
EVVAATPTSLLISWRHPHFPTRYYRITYGETGGNSPVQEFTVPLQPPTAT
ISGLKPGVDYTITGYAVTDGQNGRLLNVPISINYRT
SEQ ID NO: 32
EVVAATPTSLLISWRHHPHFPTRYYRITYGETGGNSPVQEFTVPLQPPTA
TISGLKPGVDYTITGYAVTVHWNGRELMTPISINYRT
SEQ ID NO: 33
EVVAATPTSLLISWRHPHFPTRYYRITYGETGGNSPVQEFTVPLQPPTAT
ISGLKPGVDYTITGYAVTEEWNGRVLMTPISINYRT
SEQ ID NO: 34
EVVAATPTSLLISWRHPHFPTRYYRITYGETGGNSPVQEFTVPLQPPTAT
ISGLKPGVDYTITGYAVTVERNGHTLMTPISINYRT
SEQ ID NO: 35
EVVAATPTSLLISWRHPHFPTRYYRITYGETGGNSPVQEFTVPLQPPTAT
ISGLKPGVDYTITGYAVTVEENGRQLMTPISINYRT
SEQ ID NO: 36
EVVAATPTSLLISWRHPHFPTRYYRITYGETGGNSPVQEFTVPLQPPTAT
ISGLKPGVDYTITGYAVTLERNGQVLFTPISINYRT
SEQ ID NO: 37
EVVAATPTSLLISWRHPHFPTRYYRITYGETGGNSPVQEFTVPLQPPTAT
ISGLKPGVDYTITGYAVTVERNGQVLYTPISINYRT
SEQ ID NO: 38
EVVAATPTSLLISWRHPHFPTRYYRITYGETGGNSPVQEFTVPLQPPTAT
ISGLKPGVDYTITGYAVTWGYKDHELLIPISINYRT
SEQ ID NO: 39
EVVAATPTSLLISWRHPHFPTRYYRITYGETGGNSPVQEFTVPLQPPTAT
ISGLKPGVDYTITGYAVTLGRNDRELLTPISINYRT
SEQ ID NO: 40
EVVAATPTSLLISWRHPHFPTRYYRITYGETGGNSPVQEFTVPLQPPTAT
ISGLKPGVDYTITGYAVTDGPNDRLLNIPISINYRT
SEQ ID NO: 41
EVVAATPTSLLISWRHPHFPTRYYRITYGETGGNSPVQEFTVPLQPPTAT
ISGLKPGVDYTITGYAVTFARDGHEILTPISINYRT
SEQ ID NO: 42
EVVAATPTSLLISWRHPHFPTRYYRITYGETGGNSPVQEFTVPLQPPTAT
ISGLKPGVDYTITGYAVTLEQNGRELMTPISINYRT
SEQ ID NO: 43
EVVAATPTSLLISWRHPHFPTRYYRITYGETGGNSPVQEFTVPLQPPTAT
ISGLKPGVDYTITGYAVTVEENGRVLNTPISINYRT
SEQ ID NO: 44
EVVAATPTSLLISWRHPHFPTRYYRITYGETGGNSPVQEFTVPLQPPTAT
ISGLKPGVDYTITGYAVTLEPNGRYLMVPISINYRT
SEQ ID NO: 45
EVVAATPTSLLISWRHPHFPTRYYRITYGETGGNSPVQEFTVPLQPPTAT
ISGLKPGVDYTITGYAVTEGRNGRELFIPISINYRT
SEQ ID NO: 46
VSDVPRDLEVVAATPTSLLISWRHPHFPTRYYRITYGETGGNSPVQEFTV
PLQPPAATISGLKPGVDYTITGYAVTWERNGRELFTPISINYRT
SEQ ID NO: 47
VSDVPRDLEVVAATPTSLLISWRHPHFPTRYYRITYGETGGNSPVQEFTV
PLQPPAATISGLKPGVDYTITGYAVTKERNGRELFTPISINYRT
SEQ ID NO: 48
VSDVPRDLEVVAATPTSLLISWRHPHFPTHYYRITYGETGGNSPVQEFTV
PLQPPAATISGLKPGVDYTITGYAVTTERTGRELFTPISINYRT
SEQ ID NO: 49
VSDVPRDLEVVAATPTSLLISWRHPHFPTHYYRITYGETGGNSPVQEFTV
PLQPPAATISGLKPGVDYTITGYAVTKERSGRELFTPISINYRT
SEQ ID NO: 50
VSDVPRDLEVVAATPTSLLISWRHPHFPTHYYRITYGETGGNSPVQEFTV
PLQPPAATISGLKPGVDYTITGYAVTLERDGRELFTPISINYRT
SEQ ID NO: 51
VSDVPRDLEVVAATPTSLLISWRHPHFPTRYYRITYGETGGNSPVQEFTV
PLQPPLATISGLKPGVDYTITG/VYAVTKERNGRELFTPISINYRT
SEQ ID NO: 52
VSDVPRDLEVVAATPTSLLISWRHPHFPTRYYRITYGETGGNSPVQEFTV
PLQPTTATISGLKPGVDYTITGYAVTWERNGRELFTPISINYRT
SEQ ID NO: 53
VSDVPRDLEVVAATPTSLLISWRHPHFPTRYYRITYGETGGNSPVQEFTV
PLQPTVATISGLKPGVDYTITGYAVTLERNDRELFTPISINYRT
SEQ ID NO: 54
MGEVVAATPTSLLISWRHPHFPTRYYRITYGETGGNSPVQEFTVPLQPPT
ATISGLKPGVDYTITVYAVTDGRNGRLLSIPISINYRTEIDKPSQ
SEQ ID NO: 55
MGEVVAATPTSLLISWRHPHFPTRYYRITYGETGGNSPVQEFTVPLQPPT
ATISGLKPGVDYTITVYAVTDGRNGRLLSIPISINYRTEIDKPCQ
SEQ ID NO: 56
MVSDVPRDLEVVAATPTSLLISWRHPHFPTRYYRITYGETGGNSPVQEFT
VPLQPPTATISGLKPGVDYTITVYAVTDGRNGRLLSIPISINYRTEIDKP
SQ
SEQ ID NO: 57
MGEVVAATPTSLLISWRHPHFPTRYYRITYGETGGNSPVQEFTVPLQPPT
ATISGLKPGVDYTITVYAVTDGWNGRLLSIPISINYRT
SEQ ID NO: 58
MGEVVAATPTSLLISWRHPHFPTRYYRITYGETGGNSPVQEFTVPLQPPT
ATISGLKPGVDYTITVYAVTEGPNERSLFIPISINYRT
SEQ ID NO: 59
MVSDVPRDLEVVAATPTSLLISWRHPHFPTRYYRITYGETGGNSPVQEFT
VPLQPPTATISGLKPGVDYTITVYAVTEGPNERSLFIPISINYRT

In some embodiments, the VEGFR-2 specific inhibitor is an amino acid sequence at least 80, 85, 90, 95, 98, or 100% identical to any one of SEQ ID NOs: 2-59.

In some embodiments, the VEGFR-2 specific inhibitor is a ¹⁰Fn3 based protein comprising a BC loop having the amino acid sequence set for in residues 16-23 SEQ ID NO: 4, a DE loop having the amino acid sequence set for in residues 45-49 of SEQ ID NO: 4, and an FG loop having the amino acid sequence set for in residues 70-80 of SEQ ID NO: 4.

Fibronectin based scaffold proteins include the disclosed amino acid sequences with deletions of the first 8 amino acids and may include additional amino acids at the N- or C-termini. For example, an additional MG sequence may be placed at the N-terminus. The M will usually be cleaved off, leaving a GEV . . . sequence at the N-terminus. The re-addition of the normal 8 amino acids at the N-terminus also produces a VEGFR2 binding protein with desirable properties. In some embodiments, the N-terminal methionine is cleaved off For use in vivo, a form suitable for pegylation may be generated. In one embodiment, a C-terminal tail comprising a cysteine can be added (for example, EIDKPCQ (SEQ ID NO:60) is added at the C-terminus).

### PEGylation of Fibronectin-Based Scaffold Proteins

¹⁰Fn3 polypeptides of the invention can be pegylated and retain ligand binding activity. In a preferred embodiment, the pegylated ¹⁰Fn3 polypeptide is produced by site-directed pegylation, particularly by conjugation of PEG to a cysteine moiety at the N- or C-terminus. Accordingly, the present disclosure provides a target-binding ¹⁰Fn3 polypeptide with improved pharmacokinetic properties, the polypeptide comprising: a ¹⁰Fn3 domain having from about 80 to about 150 amino acids, wherein at least one of the loops of said ¹⁰Fn3 domain participate in target binding; and a covalently bound PEG moiety, wherein said ¹⁰Fn3 polypeptide binds to the target with a K_{D} of less than 100 nM and has a clearance rate of less than 30 mL/hr/kg in a mammal. The PEG moiety may be attached to the ¹⁰Fn3 polypeptide by site directed pegylation, such as by attachment to a Cys residue, where the Cys residue may be positioned at the N-terminus of the ¹⁰Fn3 polypeptide or between the N-terminus and the most N-terminal beta or beta-like strand or at the C-terminus of the ¹⁰Fn3 polypeptide or between the C-terminus and the most C-terminal beta or beta-like strand. A Cys residue may be situated at other positions as well, particularly any of the loops that do not participate in target binding. A PEG moiety may also be attached by other chemistry, including by conjugation to amines. In addition, the invention includes this type of N or C terminal PEG conjugation to antibody moieties (e.g., camel antibodies and their derivatives, as well as single chain and domain antibodies; and particularly those expressed from microbes) and antibody-like moieties (e.g., derivatives of lipocalins, ankyrins, multiple Cys-Cys domains, and tetranectins; and particularly those expressed from microbes), particularly those less than 40 kDa that are connect by PEG, and more particularly those that have a limited number of cys amino acids.

In one specific embodiment of the present invention, modified forms of the subject soluble polypeptides comprise linking the subject soluble polypeptides to nonproteinaceous polymers. In one specific embodiment, the polymer is polyethylene glycol (“PEG”), polypropylene glycol, or polyoxyalkylenes, in the manner as set forth in U.S. Pat. No. 4,640,835; 4,496,689; 4,301,144; 4,670,417; 4,791,192 or 4,179,337. Examples of the modified polypeptide of the invention include PEGylated proteins further described herein.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ₋₁CH₂CH₂OH (1), where n is 20 to 2300 and X is H or a terminal modification, e.g., a C₁₋₄ alkyl. In one embodiment, the PEG of the invention terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, European Published Application No. 473084A and U.S. Pat. No. 5,932,462. One form of PEGs includes two PEG side-chains (PEG2) linked via the primary amino groups of a lysine (Monfardini, C., et al., Bioconjugate Chem. 6 (1995) 62-69).

In a preferred embodiment, the pegylated ¹⁰Fn3 polypeptide is produced by site-directed pegylation, particularly by conjugation of PEG to a cysteine moiety at the N- or C-terminus. Accordingly, the present disclosure provides a target-binding ¹⁰Fn3 polypeptide with improved pharmacokinetic properties, the polypeptide comprising: a ¹⁰Fn3 domain having from about 80 to about 150 amino acids, wherein at least one of the loops of said ¹⁰Fn3 domain participate in target binding; and a covalently bound PEG moiety, wherein said ¹⁰Fn3 polypeptide binds to the target with a K_{D} of less than 100 nM and has a clearance rate of less than 30 mL/hr/kg in a mammal. The PEG moiety may be attached to the ¹⁰Fn3 polypeptide by site directed pegylation, such as by attachment to a Cys residue, where the Cys residue may be positioned at the N-terminus of the ¹⁰Fn3 polypeptide or between the N-terminus and the most N-terminal beta or beta-like strand or at the C-terminus of the ¹⁰Fn3 polypeptide or between the C-terminus and the most C-terminal beta or beta-like strand. A Cys residue may be situated at other positions as well, particularly any of the loops that do not participate in target binding. A PEG moiety may also be attached by other chemistry, including by conjugation to amines.

PEG conjugation to peptides or proteins generally involves the activation of PEG and coupling of the activated PEG-intermediates directly to target proteins/peptides or to a linker, which is subsequently activated and coupled to target proteins/peptides (see Abuchowski, A. et al, J. Biol. Chem., 252, 3571 (1977) and J. Biol. Chem., 252, 3582 (1977), Zalipsky, et al., and Harris et. al., in: Poly(ethylene glycol) Chemistry: Biotechnical and Biomedical Applications; (J. M. Harris ed.) Plenum Press: New York, 1992; Chap. 21 and 22). It is noted that a binding polypeptide containing a PEG molecule is also known as a conjugated protein, whereas the protein lacking an attached PEG molecule can be referred to as unconjugated.

A variety of molecular mass forms of PEG can be selected, e.g., from about 1,000 Daltons (Da) to 100,000 Da (n is 20 to 2300), for conjugating to binding polypeptides of the invention. The number of repeating units “n” in the PEG is approximated for the molecular mass described in Daltons. It is preferred that the combined molecular mass of PEG on an activated linker is suitable for pharmaceutical use. Thus, in one embodiment, the molecular mass of the PEG molecules does not exceed 100,000 Da. For example, if three PEG molecules are attached to a linker, where each PEG molecule has the same molecular mass of 12,000 Da (each n is about 270), then the total molecular mass of PEG on the linker is about 36,000 Da (total n is about 820). The molecular masses of the PEG attached to the linker can also be different, e.g., of three molecules on a linker two PEG molecules can be 5,000 Da each (each n is about 110) and one PEG molecule can be 12,000 Da (n is about 270).

In a specific embodiment of the invention, a VEGFR2 or other receptor binding polypeptide is covalently linked to one poly(ethylene glycol) group of the formula: —CO—(CH₂)ₓ—(OCH₂CH₂)ₘ—OR, with the —CO (i.e. carbonyl) of the poly(ethylene glycol) group forming an amide bond with one of the amino groups of the binding polypeptide; R being lower alkyl; x being 2 or 3; m being from about 450 to about 950; and n and m being chosen so that the molecular weight of the conjugate minus the binding polypeptide is from about 10 to 40 kDa. In one embodiment, a binding polypeptide's c-amino group of a lysine is the available (free) amino group.

The above conjugates may be more specifically presented by formula (II): P—NHCO—(CH₂)ₓ—(OCH₂CH₂)ₘ—OR (II), wherein P is the group of a binding polypeptide as described herein, (i.e. without the amino group or amino groups which form an amide linkage with the carbonyl shown in formula (II); and wherein R is lower alkyl; x is 2 or 3; m is from about 450 to about 950 and is chosen so that the molecular weight of the conjugate minus the binding polypeptide is from about 10 to about 40 kDa. As used herein, the given ranges of “m” have an orientational meaning. The ranges of “m” are determined in any case, and exactly, by the molecular weight of the PEG group.

One skilled in the art can select a suitable molecular mass for PEG, e.g., based on how the pegylated binding polypeptide will be used therapeutically, the desired dosage, circulation time, resistance to proteolysis, immunogenicity, and other considerations. For a discussion of PEG and its use to enhance the properties of proteins, see N. V. Katre, Advanced Drug Delivery Reviews 10: 91-114 (1993).

In one embodiment of the invention, PEG molecules may be activated to react with amino groups on a binding polypeptide, such as with lysines (Bencham C. O. et al., Anal. Biochem., 131, 25 (1983); Veronese, F. M. et al., Appl. Biochem., 11, 141 (1985); Zalipsky, S. et al., Polymeric Drugs and Drug Delivery Systems, adrs 9-110 ACS Symposium Series 469 (1999); Zalipsky, S. et al., Europ. Polym. J., 19, 1177-1183 (1983); Delgado, C. et al., Biotechnology and Applied Biochemistry, 12, 119-128 (1990)).

In one specific embodiment, carbonate esters of PEG are used to form the PEG-binding polypeptide conjugates. N,N′-disuccinimidylcarbonate (DSC) may be used in the reaction with PEG to form active mixed PEG-succinimidyl carbonate that may be subsequently reacted with a nucleophilic group of a linker or an amino group of a binding polypeptide (see U.S. Pat. No. 5,281,698 and U.S. Pat. No. 5,932,462). In a similar type of reaction, 1,1′-(dibenzotriazolyl)carbonate and di-(2-pyridyl)carbonate may be reacted with PEG to form PEG-benzotriazolyl and PEG-pyridyl mixed carbonate (U.S. Pat. No. 5,382,657), respectively.

Pegylation of a ¹⁰Fn3 polypeptide can be performed according to the methods of the state of the art, for example by reaction of the binding polypeptide with electrophilically active PEGs. Preferred PEG reagents of the present invention are, e.g., N-hydroxysuccinimidyl propionates (PEG-SPA), butanoates (PEG-SBA), PEG-succinimidyl propionate or branched N-hydroxysuccinimides such as mPEG2-NHS (Monfardini, C., et al., Bioconjugate Chem. 6 (1995) 62-69). Such methods may used to pegylated at an ε-amino group of a binding polypeptide lysine or the N-terminal amino group of the binding polypeptide.

In another embodiment, PEG molecules may be coupled to sulfhydryl groups on a binding polypeptide (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. Nos. 6,610,281 and 5,766,897 describes exemplary reactive PEG species that may be coupled to sulfhydryl groups.

In some embodiments where PEG molecules are conjugated to cysteine residues on a binding polypeptide, the cysteine residues are native to the binding polypeptide, whereas in other embodiments, one or more cysteine residues are engineered into the binding polypeptide. Mutations may be introduced into a binding polypeptide coding sequence to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein. Alternatively, surface residues may be predicted by comparing the amino acid sequences of binding polypeptides, given that the crystal structure of the framework based on which binding polypeptides are designed and evolved has been solved (see Himanen et al., Nature. (2001) 20-27; 414(6866):933-8) and thus the surface-exposed residues identified. In one embodiment, cysteine residues are introduced into binding polypeptides at or near the N- and/or C-terminus, or within loop regions.

In some embodiments, the pegylated binding polypeptide comprises a PEG molecule covalently attached to the alpha amino group of the N-terminal amino acid. Site specific N-terminal reductive amination is described in Pepinsky et al., (2001) JPET, 297,1059, and U.S. Pat. No. 5,824,784. The use of a PEG-aldehyde for the reductive amination of a protein utilizing other available nucleophilic amino groups is described in U.S. Pat. No. 4,002,531, in Wieder et al., (1979) J. Biol. Chem. 254,12579, and in Chamow et al., (1994) Bioconjugate Chem. 5, 133.

In another embodiment, pegylated binding polypeptide comprises one or more PEG molecules covalently attached to a linker, which in turn is attached to the alpha amino group of the amino acid residue at the N-terminus of the binding polypeptide. Such an approach is disclosed in U.S. Publication No. 2002/0044921 and PCT Publication No. WO94/01451.

In one embodiment, a binding polypeptide is pegylated at the C-terminus. In a specific embodiment, a protein is pegylated at the C-terminus by the introduction of C-terminal azido-methionine and the subsequent conjugation of a methyl-PEG-triarylphosphine compound via the Staudinger reaction. This C-terminal conjugation method is described in Cazalis et al., C-Terminal Site-Specific PEGylation of a Truncated Thrombomodulin Mutant with Retention of Full Bioactivity, Bioconjug Chem. 2004; 15(5):1005-1009.

Monopegylation of a binding polypeptide can also be produced according to the general methods described in PCT Publication No. WO94/01451. WO94/01451 describes a method for preparing a recombinant polypeptide with a modified terminal amino acid alpha-carbon reactive group. The steps of the method involve forming the recombinant polypeptide and protecting it with one or more biologically added protecting groups at the N-terminal alpha-amine and C-terminal alpha-carboxyl. The polypeptide can then be reacted with chemical protecting agents to selectively protect reactive side chain groups and thereby prevent side chain groups from being modified. The polypeptide is then cleaved with a cleavage reagent specific for the biological protecting group to form an unprotected terminal amino acid alpha-carbon reactive group. The unprotected terminal amino acid alpha-carbon reactive group is modified with a chemical modifying agent. The side chain protected terminally modified single copy polypeptide is then deprotected at the side chain groups to form a terminally modified recombinant single copy polypeptide. The number and sequence of steps in the method can be varied to achieve selective modification at the N- and/or C-terminal amino acid of the polypeptide.

The ratio of a binding polypeptide to activated PEG in the conjugation reaction can be from about 1:0.5 to 1:50, between from about 1:1 to 1:30, or from about 1:5 to 1:15. Various aqueous buffers can be used in the present method to catalyze the covalent addition of PEG to the binding polypeptide. In one embodiment, the pH of a buffer used is from about 7.0 to 9.0. In another embodiment, the pH is in a slightly basic range, e.g., from about 7.5 to 8.5. Buffers having a pKa close to neutral pH range may be used, e.g., phosphate buffer. Other ratios will be used when making multi-specific PEG linked proteins, such as about 1:4 to 1:8, or about 1:3 to 1:5

Conventional separation and purification techniques known in the art can be used to purify PEGylated binding polypeptide, such as size exclusion (e.g., gel filtration) and ion exchange chromatography. Products may also be separated using SDS-PAGE. Products that may be separated include mono-, di-, tri- poly- and un-pegylated binding polypeptide, as well as free PEG. The percentage of mono-PEG conjugates can be controlled by pooling broader fractions around the elution peak to increase the percentage of mono-PEG in the composition. About ninety percent mono-PEG conjugates represents a good balance of yield and activity. Compositions in which, for example, at least ninety-two percent or at least ninety-six percent of the conjugates are mono-PEG species may be desired. In an embodiment of this invention the percentage of mono-PEG conjugates is from ninety percent to ninety-six percent.

In one embodiment, PEGylated binding polypeptides of the invention contain one, two or more PEG moieties. In one embodiment, the PEG moiety(ies) are bound to an amino acid residue which is on the surface of the protein and/or away from the surface that contacts the target ligand. In one embodiment, the combined or total molecular mass of PEG in PEG-binding polypeptide is from about 3,000 Da to 60,000 Da, optionally from about 10,000 Da to 36,000 Da. In a one embodiment, the PEG in pegylated binding polypeptide is a substantially linear, straight-chain PEG.

In one embodiment of the invention, the PEG in pegylated binding polypeptide is not hydrolyzed from the pegylated amino acid residue using a hydroxylamine assay, e.g., 450 mM hydroxylamine (pH 6.5) over 8 to 16 hours at room temperature, and is thus stable. In one embodiment, greater than 80% of the composition is stable mono-PEG-binding polypeptide, more preferably at least 90%, and most preferably at least 95%.

In another embodiment, the pegylated binding polypeptides of the invention will preferably retain at least about 25%, 50%, 60%, 70%, 80%, 85%, 90%, 95% or 100% of the biological activity associated with the unmodified protein. In one embodiment, biological activity refers to its ability to bind to VEGFR-2, as assessed by K_{D}, kₒₙ or k_{off}. In one specific embodiment, the pegylated binding polypeptide protein shows an increase in binding to VEGFR2 relative to unpegylated binding polypeptide.

The serum clearance rate of PEG-modified polypeptide may be decreased by about 10%, 20%, 30%, 40%, 50%, 60%, 70%, 80%, or even 90%, relative to the clearance rate of the unmodified binding polypeptide. The PEG-modified polypeptide may have a half-life (t_{1/2}) which is enhanced relative to the half-life of the unmodified protein. The half-life of PEG-binding polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified binding polypeptide. In some embodiments, the protein half-life is determined in vitro, such as in a buffered saline solution or in serum. In other embodiments, the protein half-life is an in vivo half life, such as the half-life of the protein in the serum or other bodily fluid of an animal.

mTOR Inhibitors

Mammalian target of rapamycin (“mTOR”) regulates the activity of at least two proteins involved in the translation of specific cell cycle regulatory proteins. One of these proteins, p70s6 kinase, is phosphorylated by mTOR on serine 389 as well as threonine 412. This phosphorylation can be observed in growth factor treated cells by Western blotting of whole cell extracts of these cells with antibody specific for the phosphoserine 389 residue. As used herein, the term “mTOR inhibitor” means a compound or ligand which inhibits cell replication by blocking progression of the cell cycle from G1 to S by inhibiting the phosphorylation of serine 389 of p70s6 kinase by mTOR. One skilled in the art can readily determine if a compound, such as a rapamycin derivative, is an mTOR inhibitor. A specific method of making such determination is disclosed in U.S. Publication No. 2003/0008923, the disclosure of which is incorporated herein by reference in its entirety.

Examples of mTOR inhibitors include, without limitation, rapamycin (sirolimus), rapamycin derivatives, CI-779, everolimus (Certican™), ABT-578, tacrolimus (FK 506), ABT-578, AP-23675, BEZ-235, OSI-027, QLT-0447, ABI-009, BC-210, salirasib, TAFA-93, deforolimus (AP-23573), and AP-23841. In a preferred embodiment, the mTOR inhibitor is temsirolimus (Torisel™).

### Therapeutic Uses

The present invention provides methods of treating a neoplasm in a subject in need thereof including administering to the patient at least one VEGFR2 specific inhibitor together or in parallel with at least one mTOR inhibitor in amounts that together are effective to treat said neoplasm. Neoplasia disorders include, but are not limited to, acral lentiginous melanoma, actinic keratoses, adenocarcinoma, adenoid cycstic carcinoma, adenomas, adenosarcoma, adenosquamous carcinoma, adrenocortical carcinoma, AIDS-related lymphoma, anal cancer, astrocytic tumors, bartholin gland carcinoma, basal cell carcinoma, biliary tract cancer, bone cancer, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cervical cancer, connective tissue cancer, cholangiocarcinoma, chondosarcoma, choriod plexus papilloma/carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, eye cancer, fibrolamellar, focal nodular hyperplasia, gallbladder cancer, gastric cancer, gastrinoma, germ cell tumors, gestational trophoblastic tumor, glioblastoma, glioma, glucagonoma, head and neck cancer, hemangiblastomas, hemangioendothelioma, hemangiomas, hepatic adenoma, hepatic adenomatosis, hepatocellular carcinoma, leukemias including but not limited to acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to chronic myelocytic (granulocytic) leukemia, chronic lymphocytic leukemia, hairy cell leukemia, lymphomas such as non-Hodgkin's lymphoma and Hodgkin's lymphoma, hypopharyngeal cancer, hypothalamic and visual pathway glioma, childhood, insulinoma, intaepithelial neoplasia, interepithelial squamous cell neoplasia, intraocular melanoma, intra-epithelial neoplasm, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-small cell lung cancer, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, cancer of the respiratory system, retinoblastoma, rhabdomyosarcoma, sarcoma, serous carcinoma, skin cancer, small cell carcinoma, small intestine cancer, soft tissue carcinomas, somatostatin-secreting tumor, squamous carcinoma, squamous cell carcinoma, stomach cancer, stromal tumors, submesothelial, superficial spreading melanoma, supratentorial primitive neuroectodermal tumors, testicular cancer, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

Another aspect of the invention provides methods of reducing the severity, delaying the onset, or preventing the development of VEGFR2 resistance in a subject afflicted with a neoplasm comprising administering at least one VEGFR2 specific inhibitor together or in parallel with at least one mTOR inhibitor. In some embodiments, the severity of VEGF2 resistance is reduced by at least 25%. In some embodiments, VEGFR2 resistance is delayed by at least 1, 2, 3, 4, 5, 6, 8, 12, 24, 36, 48 weeks or more.

### Formulation and Administration

Therapeutic formulations of the invention are prepared for storage by mixing the described inhibitors having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes. The formulations are preferably pyrogen free.

Sustained-release preparations may be prepared. Suitable examples of sustained-release preparations include semipermeable matrices of solid hydrophobic polymers containing the proteins of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins of the invention may remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

While the skilled artisan will understand that the dosage of each therapeutic agent will be dependent on the identity of the agent, the preferred dosages can range from about 10 mg/square meter to about 2000 mg/square meter, more preferably from about 50 mg/square meter to about 1000 mg/square meter.

For therapeutic applications, the proteins or conjugates of the invention are administered to a subject, in a pharmaceutically acceptable dosage form. They can be administered intravenously as a bolus or by continuous infusion over a period of time, by intramuscular, subcutaneous, intra-articular, intrasynovial, intrathecal, oral, topical, or inhalation routes. The protein may also be administered by intratumoral, peritumoral, intralesional, or perilesional routes, to exert local as well as systemic therapeutic effects. Suitable pharmaceutically acceptable carriers, diluents, and excipients are well known and can be determined by those of skill in the art as the clinical situation warrants. Examples of suitable carriers, diluents and/or excipients include: (1) Dulbecco's phosphate buffered saline, pH about 7.4, containing about 1 mg/ml to 25 mg/ml human serum albumin, (2) 0.9% saline (0.9% w/v NaCl), and (3) 5% (w/v) dextrose. The method of the present invention can be practiced in vitro, in vivo, or ex vivo. The inhibitors of the invention can be in the formulation in a concentration of from 1 to 15 mg/ml. In one embodiment, the formulations are administered intravenously. Suitable pharmaceutically acceptable carriers, diluents, and excipients for co-administration will be understood by the skilled artisan to depend on the identity of the particular therapeutic agent being co-administered.

When present in an aqueous dosage form, rather than being lyophilized, the inhibitor typically will be formulated at a concentration of about 0.1 mg/ml to 100 mg/ml, although wide variation outside of these ranges is permitted. For the treatment of disease, the appropriate dosage of the inhibitor will depend on the type of disease to be treated, as defined above, the severity and course of the disease, whether the inhibitors are administered for preventive or therapeutic purposes, the course of previous therapy, the patient's clinical history and response to the antibody, and the discretion of the attending physician. The inhibitors are suitably administered to the patient at one time or over a series of treatments.

Depending on the type and severity of the disease, preferably from about 1 mg/square meter to about 2000 mg/square meter of inhibitor is an initial candidate dosage for administration to the patient, more preferably from about 10 mg/square meter to about 1000 mg/square meter of inhibitor whether, for example, by one or more separate administrations, or by continuous infusion. For repeated administrations over several days or longer, depending on the condition, the treatment is repeated until a desired suppression of disease symptoms occurs. However, other dosage regimens may be useful and are not excluded.

In some embodiments, the inhibitors of the invention are subcutaneously administered. The inhibitors are formulated into pharmaceutically acceptable compositions and may be administered twice daily, once daily, on alternative days, or weekly. In some embodiments, the inhibitors are administered between 0.5 mg/kg to 2 mg/kg. In some embodiments, the inhibitors are administered at 0.1, 0.2, 0.3, or 0.4 mg/kg daily. In some embodiments, the patient is first administered an IV load of inhibitor, for example from 0.5 to 2 mg/kg.

The VEGFR2 specific inhibitor and mTOR inhibitor are administered to a patient conjointly. The inhibitors may be administered in parallel, i.e., they are administered is separate pharmaceutical compositions. They may be administered at the same time or sequentially. The dosage schedule of the inhibitors may be different, although overlapping in time. Alternatively, the inhibitors may be administered together, i.e., in a single pharmaceutical composition. In some embodiments, the mTOR and VEGFR2 specific inhibitors are administered in parallel within five days of each other, 24 hours, 12 hours, or 6 hours of each other.

The present invention also includes kits comprising a VEGFR2 specific inhibitor and an mTOR inhibitor, and instructions for the use thereof. The instructions include instructions for inhibiting the growth of a cancer cell using the combination of the invention and/or instructions for a method of treating a patient having a cancer using combination.

The elements of the kits of the present invention are in a suitable form for a kit, such as a solution or lyophilized powder. The concentration or amount of the elements of the kits will be understood by the skilled artisan to varying depending on the identity and intended use of each element of the kit.

When a kit is supplied, the different components of the combination may be packaged in separate containers and admixed immediately before use. Such packaging of the components separately may permit long-term storage without losing the active components' functions. The inhibitors may be present a single container.

The reagents included in the kits can be supplied in containers of any sort such that the life of the different components are preserved and are not adsorbed or altered by the materials of the container. For example, sealed glass ampules may contain lyophilized therapeutic agents, or buffers that have been packaged under a neutral, non-reacting gas, such as nitrogen. Ampules may consist of any suitable material, such as glass, organic polymers, such as polycarbonate, polystyrene, etc., ceramic, metal or any other material typically employed to hold similar reagents. Other examples of suitable containers include simple bottles that may be fabricated from similar substances as ampules, and envelopes, that may comprise foil-lined interiors, such as aluminum or an alloy. Other containers include test tubes, vials, flasks, bottles, IV bags, syringes, or the like. Containers may have a sterile access port, such as a bottle having a stopper that can be pierced by a hypodermic injection needle. Other containers may have two compartments that are separated by a readily removable membrane that upon removal permits the components to be mixed. Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, flash memory device etc. Detailed instructions may not be physically associated with the kit; instead, a user may be directed to an interne web site specified by the manufacturer or distributor of the kit, or supplied as electronic mail.

The cancers and cells there from referred to in the instructions of the kits include breast cancer, colon cancer, ovarian carcinoma, osteosarcoma, cervical cancer, prostate cancer, lung cancer, synovial carcinoma, pancreatic cancer, melanoma, multiple myeloma, neuroblastoma, and rhabdomyosarcoma.

### Incorporation by Reference

All documents and references, including patent documents and websites, described herein are individually incorporated by reference to into this document to the same extent as if there were written in this document in full or in part.

### EXAMPLES

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### Example 1

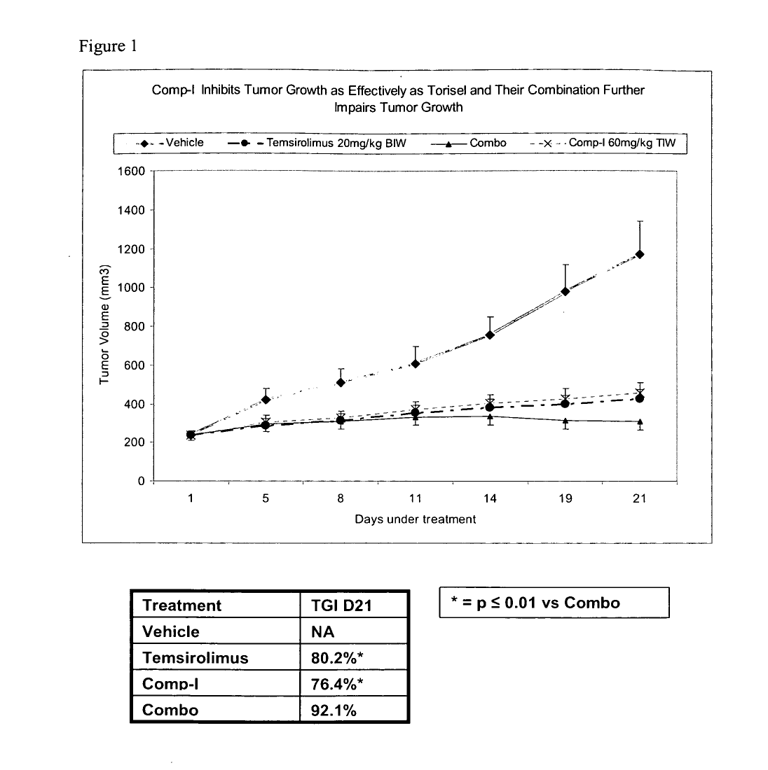

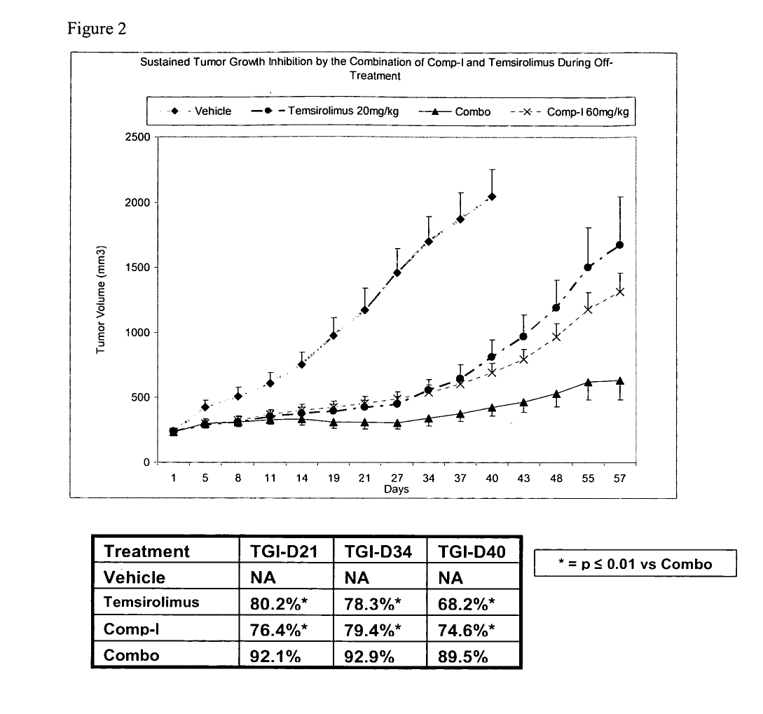

Human Colo205 cells were injected subcutaneously into the flanks of nude mice. Mice were randomized and treatments initiated when tumors reached an average size of 235 mm³. Optimal doses of Comp-I (the VEGFR2 specific inhibitor represented by SEQ ID NO: 4) and Temsilorimus were administered intraperitoneally to Colo-205 tumor bearing-mice in doses and schedules as indicated in Table 1. Tumor growth was evaluated for 21 days under treatment (FIG. 1). Mice were taken off treatment after day 21 until day 57, with a continuous assessment of tumor growth (FIG. 2). Control was euthanized on Day 41.

TABLE 1
Experimental design to evaluate Comp-I vs. Temsirolimus

| Group | Treatment (n = 12) | Dose(mg/kg) | Dose Schedule |
|---|---|---|---|
| 1 | Vehicle | NA | TIW |
| 2 | Comp-I | 60 | TIW |
| 3 | Temsirolimus | 20 | BIW |
| 4 | Comp-I/Temsirolimus | 60/20 | TIW/BIW |

Tumors were measured with calipers twice a week. Tumor volumes (mm³) were calculated by the formula [π/6 (L×W²)], where L represents the largest tumor diameter (mm) and W represents the smallest tumor diameter (mm). Tumor measurements were noted as absolute values. Animal body weights were determined weekly during the course of the experiment. Tumor Growth Inhibition (TGI) was calculated as the percent tumor growth of treated (T) groups from control group (vehicle, C). Tumor growth was calculated by subtracting initial tumor volume (at day 0) from the final tumor volume at the end of the experiment. TGI=(1−[T-T₀]/[C-C₀])*100.

TABLE 2
Antitumor Effects of Comp-I, Temsirolimus, and Combo (combination
of Comp-I and Temsirolimus) in a Colo205 xenograft model

| | Days | | | | | | |
|---|---|---|---|---|---|---|---|
| Treatment | 1 | 5 | 8 | 11 | 14 | 19 | 21 |
| Tumor Volume Average | | | | | | | |
| Vehicle | 235.21 | 421.89 | 509.80 | 609.33 | 753.80 | 976.65 | 1170.41 |
| Temsirolimus 20 mg/kg BIW | 237.19 | 287.19 | 313.73 | 354.14 | 378.44 | 395.95 | 422.42 |
| Combo | 235.50 | 296.57 | 309.75 | 331.78 | 333.70 | 313.49 | 308.96 |
| Comp-I 60 mg/kg TIW | 235.25 | 301.95 | 325.42 | 369.21 | 403.32 | 426.71 | 455.84 |
| Tumor Volume STDError | | | | | | | |
| Vehicle | 23.98 | 54.86 | 71.67 | 86.96 | 97.05 | 138.67 | 170.99 |
| Temsirolimus 20 mg/kg | 23.98 | 36.00 | 37.74 | 42.92 | 44.87 | 53.42 | 54.80 |
| Combo | 23.90 | 40.12 | 41.66 | 43.18 | 43.34 | 44.09 | 45.47 |
| Comp-I 60 mg/kg | 23.83 | 35.57 | 36.31 | 40.07 | 45.36 | 47.57 | 50.28 |

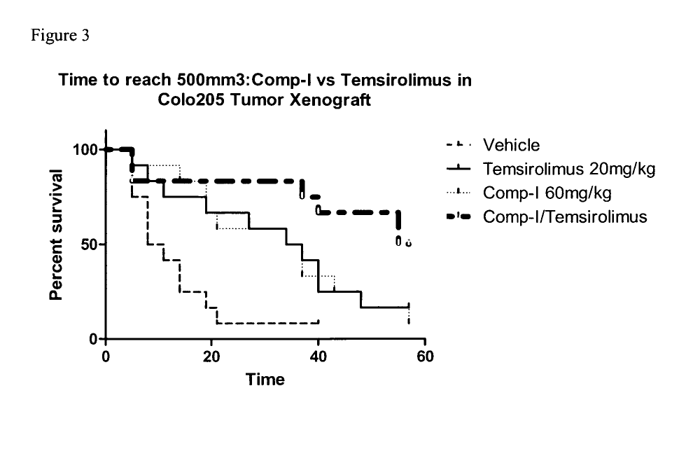

Comp-I inhibits Colo205 tumor growth to the same extent as the mTOR inhibitor Temsirolimus and their combination further impairs tumor growth (FIG. 1, Table 2). No adverse effects were observed during the treatment period. A combination of Comp-I plus Temsirolimus exhibits a sustained inhibition of Colo205 tumor growth after suspension of treatment (FIG. 2). The combinatorial inhibition of VEGFR-2 and mTOR signaling pathways synergistically enhances the antitumor activities of either agent in a Colo205 tumor model. Reduction of tumor doubling time (FIG. 3, Table 3) suggests a magnification on tumor and endothelial cell apoptosis as well as feedback inhibition on VEGF production by the mTOR antagonist activity (Temsirolimus' indirect antiangiogenic activity). Thus, these findings suggest that Comp-I and Temsirolimus together can directly maximize the blockade of the VEGF signaling pathway.

TABLE 3
Kaplan Meier Survival Analyses on FIG. 3.
Comparison of Survival Curves

| Log-rank (Mantel-Cox) Test | |
|---|---|
| Chi square | 14.91 |
| df | 3 |
| P value | 0.0019 |
| P value summary | ** |
| Are the survival curves sig different? | Yes |
| Logrank test for trend | |
| Chi square | 11.53 |
| df | 1 |
| P value | 0.0007 |
| P value summary | *** |
| Sig. trend? | Yes |

### Example 2

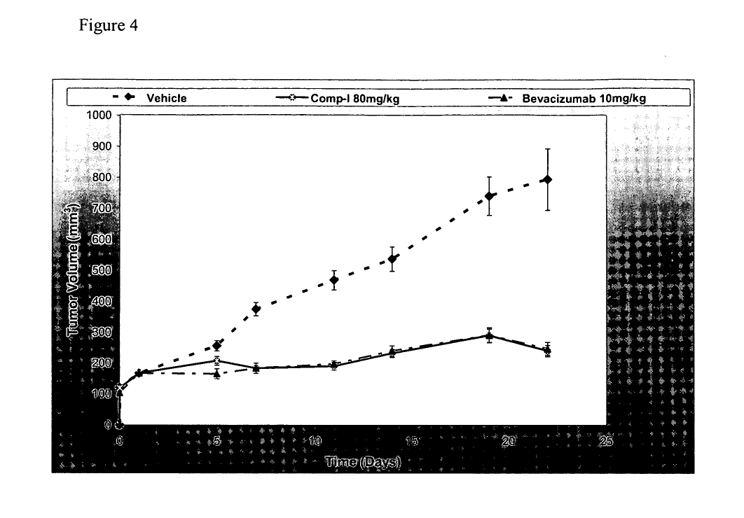

Human Colo205 cells were injected subcutaneously into the flanks of nude mice. Mice were randomized and treatments initiated when tumors reached an average size of 167 mm³. Comp-I and Bevacizumab were administered intraperitoneally to Colo-205 tumor bearing-mice in doses and schedules as indicated in Table 4. Temsirolimus treatment (10 mg/kg, BIW) was initiated on Day 17 added to Comp-I and Bevacizumab treatments on Day 17. Half the mice (n=4) in the vehicle group were initiated on torisel the same day and the other half (n=4) remained under vehicle treatment. Both anti-angiogenic treatments reduced antitumor activity to similar levels by Day 22 (FIG. 4). Percentage of tumor growth inhibition was determined as follows: Day 19/Comp-I (89%), Day 19/Bevacizumab (87%).

TABLE 4
Experimental design to evaluate Comp-I vs. Bevacizumab

| | Day 1 (TV, mm3) | Day 19 (TV, mm3) | | |
|---|---|---|---|---|
| Treatments | Average ± STDErr | Average ± STDErr | Day 22 (TV, mm3) | |
| (n = 8/group) | (Mean) | (Mean) | Average ± STDErr (Mean) | |
| 1. Vehicle | 167.3 ± 8.3 | 739 ± 62.3 | Plus mTOR inhibitor | |
| | (163.0) | (753.5) | (n = 4) | (n = 4) |
| | | | 612.3 ± 99.5 (667.5) | 975.7 ± 47.9 |
| | | | | (1008.5) |
| 2. Bevacizumab | 167.7 ± 7.7 | 290.3 ± 24.6 | 246.5 ± 21.2 (250.7) | |
| 10 mg/kg BIW | (166.7) | (299.2) | | |
| 3. Comp-I | 167.4 ± 8.1 | 289.4 ± 21.5 | 239.4 ± 18.3 (247.1) | |
| 80 mg/kg TIW | (166.2) | (293.1) | | |

### Example 3

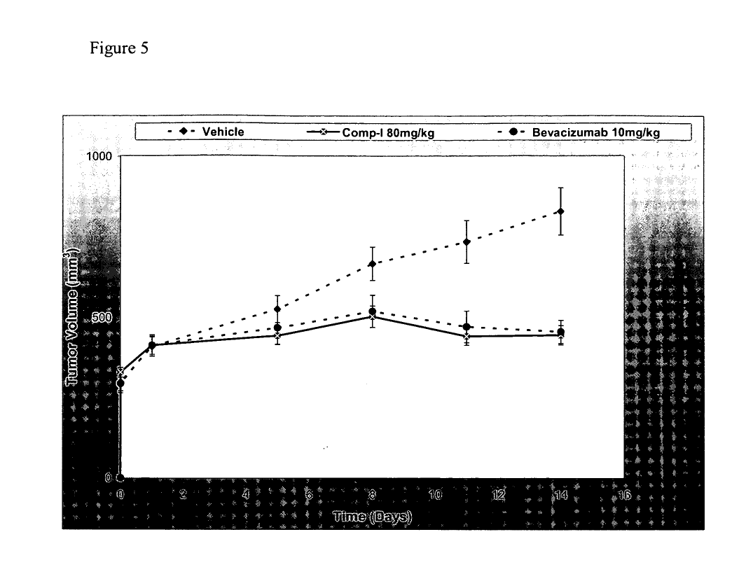

Human Colo205 cells were injected subcutaneously into the flanks of nude mice. Mice were randomized and treatments initiated when tumors reached an average size of 411 mm³. Comp-I and Bevacizumab were administered intraperitoneally to Colo-205 tumor bearing-mice in doses and schedules as indicated in Table 5. Temsirolimus (10 mg/kg, BIW) was added to Comp-I and Bevacizumab treatments on Day 9. Half the mice (n=4) in the vehicle group were initiated on torisel the same day and the other half (n=4) remained under vehicle treatment Both anti-angiogenic treatments reduced antitumor activity to similar levels by Day 14 (FIG. 5). Percentage of tumor growth inhibition was determined as follows: Day 8/Comp-I (53.03%), Day 8/Bevacizumab (38.99%), Day 11/Comp-I (91.5%), Day 11/Bevacizumab (82.2%).

TABLE 5
Experimental design to evaluate Comp-I vs. Bevacizumab

| | Day 1 (TV, mm³) | Day 8 (TV, mm³) | | |
|---|---|---|---|---|
| | Average ± | Average ± | | |
| Treatments | STDErr | STDErr | Day 14 (TV, mm³) | |
| (n = 8/group) | (Mean) | (Mean) | Average ± STDErr (Mean) | |
| 1. Vehicle | 409.13 ± 30.4 | 666.9 ± 52.1 | Plus mTOR | |
| | (413.2) | (626.4) | inhibitor | |
| | | | (n = 4) | (n = 4) |
| | | | 849.6 ± 106.6 (821.1) | 809.5 ± 128.2 (735.6) |

| | | | |
|---|---|---|---|
| 2. Bevacizumab | 413.0 ± 32.9 | 519.0 ± 50.0 | 455.1 ± 36.2 (458.9) |
| 10 mg/kg BIW | (398.6) | (511.8) | |
| 3. Comp-I | 413.5 ± 28.7 | 502.5 ± 33.6 | 444.6 ± 30.2 (449.2) |
| 80 mg/kg TIW | (417.2) | (505.8) | |

### Example 4

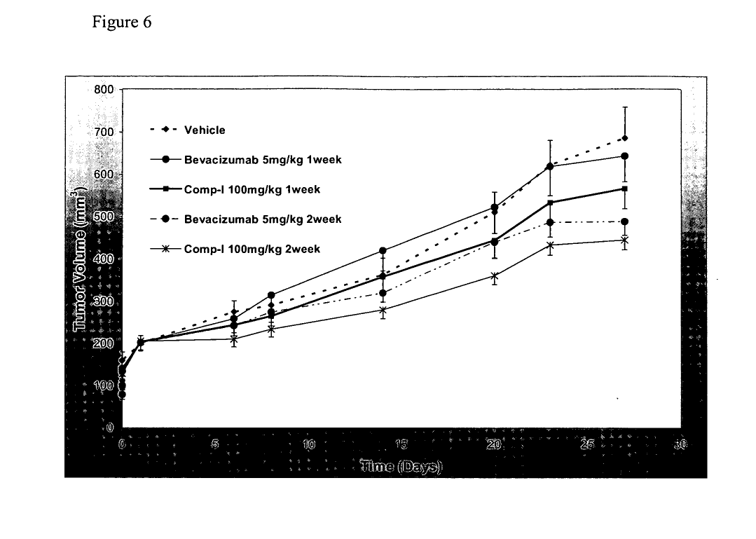

Human HT1080 cells were injected subcutaneously into the flanks of nude mice. Mice were randomized and treatments initiated when tumors reached an average size of 201 mm³. Comp-I and Bevacizumab were administered intraperitoneally to HT1080 tumor bearing-mice in doses and schedules as indicated in Table 6. Temsirolimus (10 mg/kg, BIW) was added to Comp-I and Bevacizumab treatments on Day 23. Both anti-angiogenic treatments reduced antitumor activity as measured on Day 27 (FIG. 6). However, in this model, tumor growth is more sensitive to Comp-1-mediated VEGFR-2 inhibition than to Bevacizumab-mediated VEGF inhibition.

TABLE 6
Experimental design to evaluate Comp-I vs. Bevacizumab

| | Day 1 (TV, mm³) | Day 23 (TV, mm³) |
|---|---|---|
| Treatments | Average ± STDErr | Average ± STDErr |
| (n = 8/group) | (Mean) | (Mean) |
| 1. Vehicle | 199.9 ± 18.8 (193.8) | 622.9 ± 58.5 (584.0) |
| 2. Bevacizumab 5 mg/kg QW | 202.0 ± 18.8 (195.8) | 619.3 ± 69.6 (598.3) |
| 3. Bevacizumab 5 mg/kg BIW | 201.6 ± 19.1 (191.9) | 486.9 ± 34.6 (517.4) |
| 4. Comp-I 100 mg/kg QW | 203.4 ± 18.7 (207.0) | 533.4 ± 47.9 (492.9) |
| 5. Comp-I 100 mg/kg TIW | 205.1 ± 20.9 (183.4) | 432.8 ± 23.6 (460.5) |

1. A method of treating a subject afflicted with a neoplasm, said method comprising administering to the subject a polypeptide comprising a VEGFR2-binding tenth fibronectin III domain (¹⁰Fn3) together or in parallel with at least one mTOR inhibitor in amounts that together are effective to treat said neoplasm.

2. The method of claim 1, wherein the neoplasm is a solid tumor.

3. The method of claim 1, wherein the polypeptide and the mTOR inhibitor are administered sequentially.

4. The method of claim 1, wherein the VEGFR2-binding ¹⁰Fn3 comprises a BC loop having the amino acid sequence set for in residues 16-23 SEQ ID NO: 4, a DE loop having the amino acid sequence set for in residues 45-49 of SEQ ID NO: 4, and an FG loop having the amino acid sequence set for in residues 70-80 of SEQ ID NO: 4.

5. The method of claim 1, wherein the VEGFR2-binding ¹⁰Fn3 comprises an amino acid sequence at least 90% identical to any one of SEQ ID NOS: 2-59.

6. The method of claim 1, wherein the mTOR inhibitor is temsilolimus.

7. A method of reducing the severity, delaying the onset, or preventing the development of VEGFR2 resistance in a subject afflicted with a neoplasm, said method comprising administering at a polypeptide comprising a VEGFR2-binding tenth fibronectin III domain (¹⁰Fn3) together or in parallel with at least one mTOR inhibitor.

8. The method of claim 7, wherein the neoplasm is a solid tumor.

9. The method of claim 7, wherein the polypeptide and the mTOR inhibitor are administered sequentially.

10. The method of claim 7, wherein the VEGFR2-binding ¹⁰Fn3 comprises a BC loop having the amino acid sequence set for in residues 16-23 SEQ ID NO: 4, a DE loop having the amino acid sequence set for in residues 45-49 of SEQ ID NO: 4, and an FG loop having the amino acid sequence set for in residues 70-80 of SEQ ID NO: 4.

11. The method of claim 7, wherein the VEGFR2-binding ¹⁰Fn3 comprises an amino acid sequence at least 90% identical to any one of SEQ ID NOS: 2-59.

12. The method of claim 8, wherein the mTOR inhibitor is temsilolimus.

13. The method of claim 8, wherein the development of VEGFR2 resistance is delayed by at least one week.


SEQUENCE LISTING (60 sequences)

SEQ ID NO: 1 - protein, 94 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 94 aa, Artificial Sequence
  source: /note=“Description of Artificial Sequence Synthetic peptide”
VSDVPRDLEVVAATPTSLLISWRHPHFPTRYYRITYGETGGNSPVQEFTVPLQPPTATISGLKPGVDYTITVYAVTEGPNERSLFIPISINYRT

SEQ ID NO: 3 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITVYAVTEGPNERSLFIPISINYRT

SEQ ID NO: 4 - protein, 94 aa, Artificial Sequence
  source: /note=“Description of Artificial Sequence Synthetic peptide”
GEVVAATPTSLLISWRHPHFPTRYYRITYGETGGNSPVQEFTVPLQPPTATISGLKPGVDYTITVYAVTDGRNGRLLSIPISINYRTEIDKPCQ

SEQ ID NO: 5 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITVYAVTDGRNGRLLSIPISINYRT

SEQ ID NO: 6 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTMGLYGHELLTPISINYRT

SEQ ID NO: 7 - protein, 86 aa, Artificial Sequence
  source: /note=“Description of Artificial Sequence Synthetic Peptide”
EVVAATPTSLLISWRHPHFPTRYYRITYGETGGNSPVQEFTVPLQPPTATISGLKPGVDYTITGYAVTDGENGQFLLVPISINYRT

SEQ ID NO: 8 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTMGPNDNELLTPISINYRT

SEQ ID NO: 9 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTAGWDDHELFIPISINYRT

SEQ ID NO: 10 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTSGHNDHMLMIPISINYRT

SEQ ID NO: 11 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTAGYNDQILMTPISINYRT

SEQ ID NO: 12 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTFGLYGKELLIPISINYRT

SEQ ID NO: 13 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTTGPNDRLLFVPISINYRT

SEQ ID NO: 14 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTDVYNDHEIKTPISINYRT

SEQ ID NO: 15 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTDGKDGRVLLTPISINYRT

SEQ ID NO: 16 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTEVHHDREIKTPISINYRT

SEQ ID NO: 17 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTQAPNDRVLYTPISINYRT

SEQ ID NO: 18 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTREENDHELLIPISINYRT

SEQ ID NO: 19 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVTHNGHPLMTPISINYRT

SEQ ID NO: 20 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTLALKGHELLTPISINYRT

SEQ ID NO: 21 - protein, 94 aa, Artificial Sequence
  source: /note=“Description of Artificial Sequence Synthetic peptide”
VSDVPRDLEVVAATPTSLLISWRHPHFPTRYYRITYGETGGNSPVQEFTVPLQPPTATISGLKPGVDYTITGYAVTVAQNDHELITPISINYRT

SEQ ID NO: 22 - protein, 94 aa, Artificial Sequence
  source: /note=“Description of Artificial Sequence Synthetic peptide”
VSDVPRDLEVVAATPTSLLISWRHPHFPTRYYRITYGETGGNSPVQEFTVPLQPPAATISGLKPGVDYTITGYAVTMAQSGHELFTPISINYRT

SEQ ID NO: 23 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVERNGRVLMTPISINYRT

SEQ ID NO: 24 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVERNGRHLMTPISINYRT

SEQ ID NO: 25 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTLERNGRELMTPISINYRT

SEQ ID NO: 26 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTEERNGRTLRTPISINYRT

SEQ ID NO: 27 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVERNDRVLFTPISINYRT

SEQ ID NO: 28 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVERNGRELMTPISINYRT

SEQ ID NO: 29 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTLERNGRELMVPISINYRT

SEQ ID NO: 30 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTDGRNDRKLMVPISINYRT

SEQ ID NO: 31 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTDGQNGRLLNVPISINYRT

SEQ ID NO: 32 - protein, 87 aa, Artificial Sequence
  source: /note=“Description of Artificial Sequence Synthetic peptide”
EVVAATPTSLLISWRHHPHFPTRYYRITYGETGGNSPVQEFTVPLQPPTATISGLKPGVDYTITGYAVTVHWNGRELMTPISINYRT

SEQ ID NO: 33 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTEEWNGRVLMTPISINYRT

SEQ ID NO: 34 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVERNGHTLMTPISINYRT

SEQ ID NO: 35 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVEENGRQLMTPISINYRT

SEQ ID NO: 36 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTLERNGQVLFTPISINYRT

SEQ ID NO: 37 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVERNGQVLYTPISINYRT

SEQ ID NO: 38 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTWGYKDHELLIPISINYRT

SEQ ID NO: 39 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTLGRNDRELLTPISINYRT

SEQ ID NO: 40 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTDGPNDRLLNIPISINYRT

SEQ ID NO: 41 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTFARDGHEILTPISINYRT

SEQ ID NO: 42 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTLEQNGRELMTPISINYRT

SEQ ID NO: 43 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTVEENGRVLNTPISINYRT

SEQ ID NO: 44 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTLEPNGRYLMVPISINYRT

SEQ ID NO: 45 - protein, 86 aa, Artificial Sequence
  source: /note=“Description of Artificial Sequence Synthetic peptide”
EVVAATPTSLLISWRHPHFPTRYYRITYGETGGNSPVQEFTVPLQPPTATISGLKPGVDYTITGYAVTEGRNGRELFIPISINYRT

SEQ ID NO: 46 - protein, 94 aa, Artificial Sequence
  source: /note=“Description of Artificial Sequence Synthetic peptide”
VSDVPRDLEVVAATPTSLLISWRHPHFPTRYYRITYGETGGNSPVQEFTVPLQPPAATISGLKPGVDYTITGYAVTWERNGRELFTPISINYRT

SEQ ID NO: 47 - protein, 94 aa, Artificial Sequence
  source: /note=“Description of Artificial Sequence Synthetic peptide”
VSDVPRDLEVVAATPTSLLISWRHPHFPTRYYRITYGETGGNSPVQEFTVPLQPPAATISGLKPGVDYTITGYAVTKERNGRELFTPISINYRT

SEQ ID NO: 48 - protein, 94 aa, Artificial Sequence
  source: /note=“Description of Artificial Sequence Synthetic peptide”
VSDVPRDLEVVAATPTSLLISWRHPHFPTHYYRITYGETGGNSPVQEFTVPLQPPAATISGLKPGVDYTITGYAVTTERTGRELFTPISINYRT

SEQ ID NO: 49 - protein, 94 aa, Artificial Sequence
  source: /note=“Description of Artificial Sequence Synthetic peptide”
VSDVPRDLEVVAATPTSLLISWRHPHFPTHYYRITYGETGGNSPVQEFTVPLQPPAATISGLKPGVDYTITGYAVTKERSGRELFTPISINYRT

SEQ ID NO: 50 - protein, 94 aa, Artificial Sequence
  source: /note=“Description of Artificial Sequence Synthetic peptide”
VSDVPRDLEVVAATPTSLLISWRHPHFPTHYYRITYGETGGNSPVQEFTVPLQPPAATISGLKPGVDYTITGYAVTLERDGRELFTPISINYRT

SEQ ID NO: 51 - protein, 94 aa, Artificial Sequence
  source: /note=“Description of Artificial Sequence Synthetic peptide”
VSDVPRDLEVVAATPTSLLISWRHPHFPTRYYRITYGETGGNSPVQEFTVPLQPPLATISGLKPGVDYTITGYAVTKERNGRELFTPISINYRT

SEQ ID NO: 52 - protein, 94 aa, Artificial Sequence
  source: /note=“Description of Artificial Sequence Synthetic peptide”
VSDVPRDLEVVAATPTSLLISWRHPHFPTRYYRITYGETGGNSPVQEFTVPLQPTTATISGLKPGVDYTITGYAVTWERNGRELFTPISINYRT

SEQ ID NO: 53 - protein, 94 aa, Artificial Sequence
  source: /note=“Description of Artificial Sequence Synthetic peptide”
VSDVPRDLEVVAATPTSLLISWRHPHFPTRYYRITYGETGGNSPVQEFTVPLQPTVATISGLKPGVDYTITGYAVTLERNDRELFTPISINYRT

SEQ ID NO: 54 - protein, 95 aa, Artificial Sequence
  source: /note=“Description of Artificial Sequence Synthetic peptide”
MGEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 55 - protein, 95 aa, Artificial Sequence
  source: /note=“Description of Artificial Sequence Synthetic peptide”
MGEVVAATPTSLLISWRHPHFPTRYYRITYGETGGNSPVQEFTVPLQPPTATISGLKPGVDYTITVYAVTDGRNGRLLSIPISINYRTEIDKPCQ

SEQ ID NO: 56 - protein, 102 aa, Artificial Sequence
  source: /note=“Description of Artificial Sequence Synthetic polypeptide”
MVSDVPRDLEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 57 - protein, 88 aa, Artificial Sequence
  source: /note=“Description of Artificial Sequence Synthetic peptide”
MGEVVAATPTSLLISWRHPHFPTRYYRITYGETGGNSPVQEFTVPLQPPTATISGLKPGVDYTITVYAVTDGWNGRLLSIPISINYRT

SEQ ID NO: 58 - protein, 88 aa, Artificial Sequence
  source: /note=“Description of Artificial Sequence Synthetic peptide”
MGEVVAATPTSLLISWRHPHFPTRYYRITYGETGGNSPVQEFTVPLQPPTATISGLKPGVDYTITVYAVTEGPNERSLFIPISINYRT

SEQ ID NO: 59 - protein, 95 aa, Artificial Sequence
  source: /note=“Description of Artificial Sequence Synthetic peptide”
MVSDVPRDLEVVAATPTSLLISWRHPHFPTRYYRITYGETGGNSPVQEFTVPLQPPTATISGLKPGVDYTITVYAVTEGPNERSLFIPISINYRT

SEQ ID NO: 60 - protein, 7 aa, Artificial Sequence
  source: /note=“Description of Artificial Sequence Synthetic peptide”
EIDKPCQ